(19) United States
(12) Patent Application Publication
(10) Pub. No.: US 2022/0054838 A1
(43) Pub. Date: Feb. 24, 2022

(54) IMPLANTABLE NEUROSTIMULATION SYSTEM

(71) Applicants: Galvani Bioelectronics Limited, Brentford, Middlesex (GB); The Trustees of Indiana University, Bloomington, IN (US)

(72) Inventors: Michael John Carr, Wilmington, DE (US); Rizwan Bashirullah, Wilmington, DE (US); Kenichi Yoshida, Bloomington, IN (US)

(21) Appl. No.: 17/415,641

(22) PCT Filed: Dec. 13, 2019
(86) PCT No.: PCT/IB2019/060772

§ 371 Date: Jun. 17, 2021

Related U.S. Application Data
(60) Provisional application No. 62/784,008, filed on Dec. 21, 2018.

(51) Int. Cl. A61N 1/36

(52) U.S. Cl. CPC A61N 1/36053; A61N 1/36167; A61N 1/36139

(57) ABSTRACT

An implantable neurostimulation system comprises at least one neural interface device for stimulating and/or inhibiting neural activity in a nerve such as the cervical vagus nerve. The device comprises first and second electrodes and at least one signal generator configured to generate first and second electrical signals that stimulate and/or inhibit neural activity in the nerve via the first and second electrodes. The first electrical signal is configured to stimulate neural activity in the nerve to cause at least one pre-determined physiological response; and the second electrical signal is configured to inhibit neural activity in the nerve to at least partially suppress the least one pre-determined physiological response.

Activation of nerve fibers using electricity has been known since antiquity. Methods to block propagating action potentials (AP) are a more recent discovery.

Electrical nerve conduction block is useful in impeding nerve activity in a range of applications [1-5]. Previously, conduction block methods have been employed in both motor and sensory nerve fibers in attempts to address disorders caused by neural over-activity [6]. Successful electrically induced nerve conduction block could be used to stop nerve activity in peripheral nerves, velocity selective gating, and unidirectional activation. Other techniques such as direct current (DC), ramp, carousel, kilohertz frequency alternating current (KHFAC), and combinations of the aforementioned have all been documented as techniques for conduction block. Methods range drastically in the nerve of interest, waveforms utilized, quantitative measures, electrode geometry, composition, number, as well as timing. Two main drawbacks limit the clinical application of conduction block: 1) the presence of an onset response and 2) the possibility of creating reactive species (free radicals) that alter the electrode-tissue interface and reduce nerve conductivity. Reducing the effects of these drawbacks could generate more efficient method of inhibiting nerve conduction activity of the autonomic or somatic nervous system.

A major component of conduction block that must be resolved is the ‘onset response’. First characterized in 1964, this transient burst of action potentials can be especially bothersome in that it can cause patient discomfort, pain, and intense muscle contractions [7]. Usually in the mid-to-low voltage range, the onset response precedes the high voltages required in some blocking applications. Unwanted physiological responses caused by the onset response can last anywhere from ˜100 ms to tens of seconds [3]. Waveform selection is vital for mitigating the effects of the onset response. Some have been able to reduce the duration of onset symptoms substantially by altering electrode geometry [8] or nullify the response completely by adding additional electrodes with alternative blocking techniques [1]. The intricate characteristics of the onset response are described in [4].

During charge introduction, capacitive or Faradaic reactions can occur as a function of charge intensity [9]. Low levels can be repeated reliably but as intensity increases Faradaic reactions can cause reductive and oxidative mechanisms [9]. High intensity, extended duration charge injection can cause irreversible Faradic interactions leading to formation of reactive species. Reactive species cause neural damage and reduce nerve conductivity [10]. Effects can be alleviated by proper waveform selection and a suitable recharge phase with opposite polarity to the initial phase. Often, researchers will use pulse trains varying in duration from hundreds of milliseconds to minutes. Variations in electrode geometry and nerve diameter require current amplitudes to be adjusted between tens of microamperes to tens of milliamperes. Other waveform shapes have been used besides pulses due to the sharp edges producing ‘make’ and ‘break’ excitation [2]. Alternatively, the use of sinusoidal waveforms can complement the anti-polarity charge injection during the different phases of the waveform. For use of conduction block in clinical applications, the possibility of producing reactive species must be mitigated.

Reducing the frequency of the sinusoid to <100 Hz has been shown to achieve phasic blocking of action potentials in in-vivo earthworm nerve cords. This was possible at current levels that are are less of those required for kFHAC block, within the linear working range of the electrodes and with no indication of onset activation. The phenomenon showed low threshold characteristics of DC block and the charge balanced reversibility of kHFAC block in the frequency continuum gap between DC block (0 Hz) and kHFAC block (>1 kHz). Thus, it is referred to herein as low frequency alternating current (LFAC) block.

Described herein is the application of a LFAC block in-vivo on an intact mammalian nerve preparation, measuring its efficacy using functional changes to organ function as a biomarker. More specifically, described herein is a method to reversibly block nerve conduction using a low frequency (1 Hz) alternating current (LFACb) waveform. An in situ electrophysiology setup was used to assess the LFACb on propagating action potentials (APs) within the cervical vagus nerve in 6 anaesthetized Sprague-Dawley rats. Two sets of hook electrodes were used. The rostral hook was used to generate a volley of APs while the LFACb waveform was presented to the caudal hook. This efferent volley, if unblocked, elicits acute bradycardia and hypotension. Block was assessed by ability to reduce this bradycardic drive by monitoring the heart rate (HR) and blood pressure (BP) during LFACb alone, LFACb and vagal stimulation, and vagal stimulation alone. Using the LFACb technique 82±15% conduction block was achieved with current levels 100±36 μAₚ.

Accordingly, the present invention provides an implantable neurostimulation system, comprising: at least one neural interface device for stimulating and/or inhibiting neural activity in a nerve such as the cervical vagus nerve, the at least one neural interface device comprising: first and second electrodes; at least one signal generator electrically coupled to the first and second electrodes and configured to generate first and second electrical signals that stimulate and/or inhibit neural activity in the nerve with the first and second electrical signals via the first and second electrodes, respectively; wherein the first electrical signal is configured to stimulate neural activity in the nerve to cause at least one pre-determined physiological response; and wherein the second electrical signal is configured to inhibit neural activity in the nerve to at least partially, optionally fully, suppress the least one pre-determined physiological response; the implantable neurostimulation system further comprising: at least one physiological sensor to detect the at least one pre-determined physiological response; and a control system communicatively coupled to the at least one physiological sensor and configured to generate a feedback response, upon receiving a signal from at least one physiological sensor.

Preferably the control system is configured to cause the signal generator to generate the first signal to stimulate neural activity in the nerve at a first time, and to cause the signal generator to generate the second signal to inhibit neural activity in the nerve at a second time later than the first time and concomitantly with the first signal.

Alternatively, the control system is configured to cause the signal generator to generate the second signal to inhibit neural activity in the nerve at a first time, and to cause the signal generator to generate the first signal to stimulate neural activity in the nerve at a second time later than the first time and concomitantly with the second signal.

The control system may be configured to generate a feedback response upon receiving a signal from the at least one physiological sensor whilst the second signal is being generated concomitantly with the first signal.

The control system may be configured to compare a first signal comparator representative of a signal from the at least one physiological sensor whilst the second signal is being generated concomitantly with the first signal, with a second signal comparator representative of a signal from the at least one physiological sensor whilst the first signal is being generated without the second signal.

The control system may be configured to generate a feedback response based on a comparison of the first signal comparator with the second signal comparator.

The feedback response may be indicative of the effectiveness of the second electrical signal to inhibit neural activity and thus to at least partially, optionally fully, suppress the least one pre-determined physiological response. For example, the feedback response may be indicative of the pre-determined physiological response being fully suppressed. Alternatively, the feedback response may be indicative of the pre-determined physiological response being partially suppressed. Alternatively the feedback response may be indicative of the pre-determined physiological response not being suppressed.

The control system may be configured to modify the second signal (which may be an increase or decrease of any signal parameter) based on a feedback response indicative of the pre-determined physiological response being partially suppressed or not being suppressed.

The at least one pre-determined physiological response may be a change in heart rate, respiratory rate, and/or blood pressure; and the at least one physiological sensor may be a heart rate sensor, a sensor for detecting respiratory rate, and/or a blood pressure sensor, respectively.

The first electrode may be located either closer to or further away from the brain along the nerve axis than the second electrode.

The first electrical signal may comprises a pre-determined pattern that causes the pre-determined physiological response. For example, the first electrical signal may comprises a pulse train, preferably formed of rectangular pulses. Any other shaped pulses may also be used. Each pulse train may consist of between 5 and 15, preferably between 8 and 12, preferably 10 pulses. The pulses may have a pulse width of between 0.1 ms and 1.5 ms, preferably between 0.3 ms and 1.2 ms, preferably between 0.5 ms and 1 ms, preferably 1 ms. The pulse train may have a frequency of between 10 Hz and 100 Hz, preferably between 20 Hz and 80 Hz, preferably between 25 Hz and 50 Hz, preferably 25 Hz.

The second electrical signal comprises a symmetrical, preferably sinusoidal waveform. The waveform may have a frequency between 0.5 and 100 Hz, preferably between 0.6 and 50 Hz, preferably between 0.7 and 20 Hz preferably between 0.8 and 10 Hz, preferably 1 Hz. The waveform may have an amplitude between 50 μAp and 2 mA, preferably between 60 μAp and 1 mA, preferably between 70 μAp and 150 μAp, preferably between 80 and 120 μAp, preferably 100 μAp.

Preferably, the control system is configured to generate the second signal concomitantly with the first signal for a first period.

Moreover, the control system may be configured to generate the second signal without the first signal for a second period prior to the first period and/or the control system may be configured to generate the first signal without the second signal for a third period following the first period.

Alternatively, the control system may be configured to generate the second signal without the first signal for a second period following the first period and/or the control system is configured to generate the first signal without the second signal for a third period prior to the first period.

Any, some or all of the first, second and third periods may be 20 seconds duration, though other durations are possible.

The invention will now be described with reference to the figures, in which:

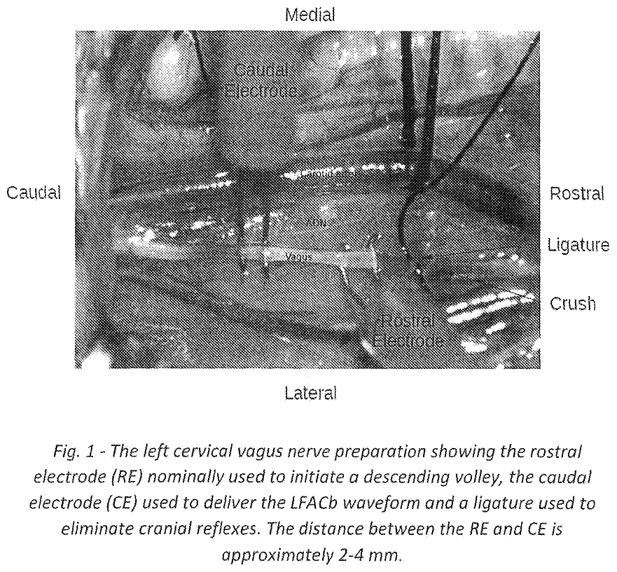

FIG. 1 is an image showing the left cervical vagus nerve preparation showing the rostral electrode (RE) nominally used to initiate a descending volley, the caudal electrode (CE) used to deliver the LFACb waveform and a ligature used to eliminate cranial reflexes. The distance between the RE and CE is approximately 2-4 mm.

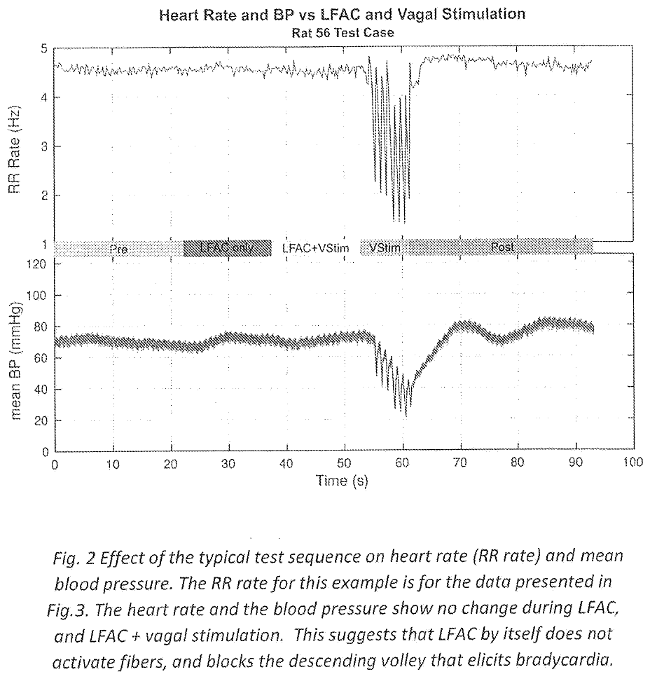

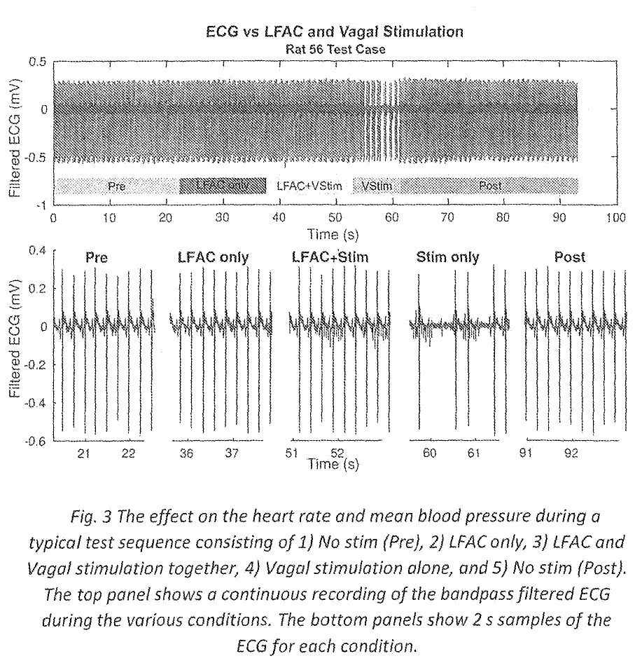

FIG. 2 is a graph showing the effect of the typical test sequence on heart rate (RR rate) and mean blood pressure. The RR rate for this example is for the data presented in FIG. 3. The heart rate and the blood pressure show no change during LFAC, and LFAC+vagal stimulation. This suggests that LFAC by itself does not activate fibers, and blocks the descending volley that elicits bradycardia.

FIG. 3 is a graph showing the effect on the heart rate and mean blood pressure during a typical test sequence consisting of 1) No stim (Pre), 2) LFAC only, 3) LFAC and Vagal stimulation together, 4) Vagal stimulation alone, and 5) No stim (Post). The top panel shows a continuous recording of the bandpass filtered ECG during the various conditions. The bottom panels show 2 s samples of the ECG for each condition.

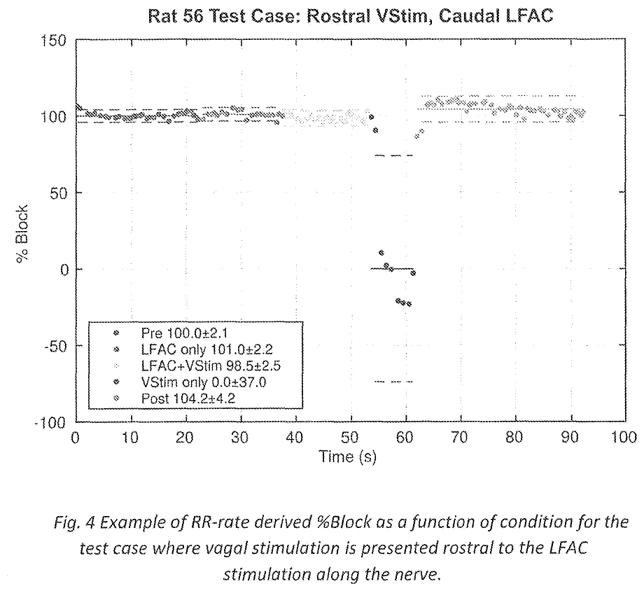

FIG. 4 is a graph showing the example of RR-rate derived % Block as a function of condition for the test case where vagal stimulation is presented rostral to the LFAC stimulation along the nerve.

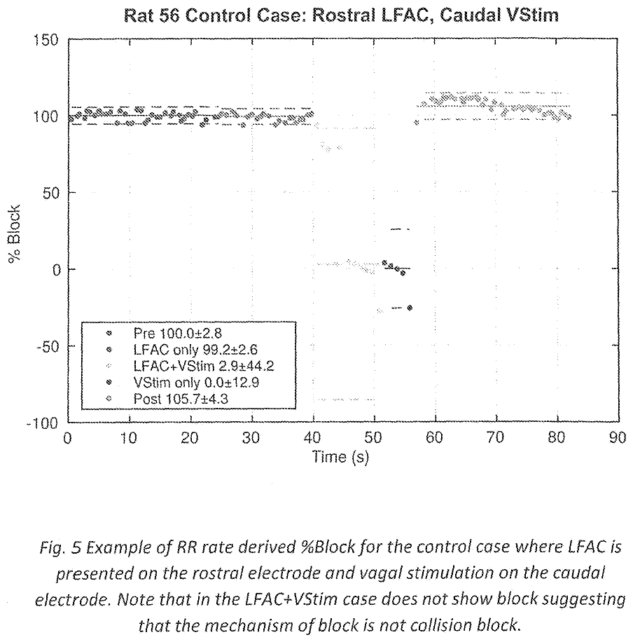

FIG. 5 is a graph showing the example of RR rate derived % Block for the control case where LFAC is presented on the rostral electrode and vagal stimulation on the caudal electrode. The graph shows that in the LFAC+VStim case, there is no showing of a block, suggesting that the mechanism of block is not collision block.

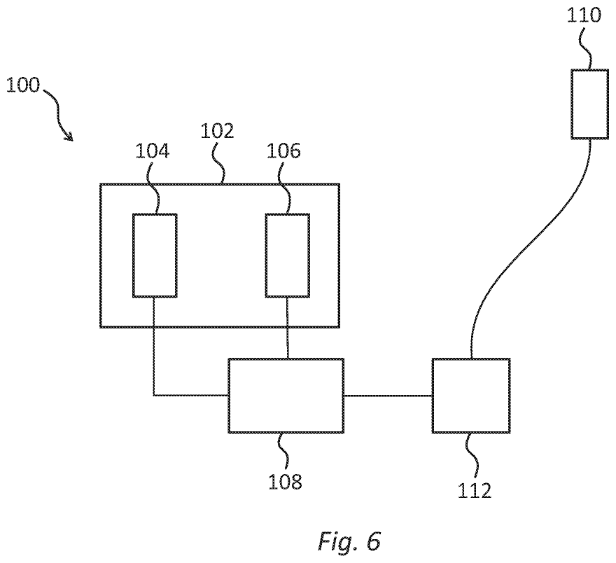

FIG. 6 is a diagram of an exemplary implantable neurostimulation system according to the invention.

### EXAMPLES

A. Animal and Surgical Preparation

All animal use protocols were approved by the Purdue School of Science Institutional Animal Care and Use Committee (SoS IACUC) at Indiana University Purdue—University Indianapolis (IUPUI). The electrophysiological preparation mirrored that of [16]. A total of 6 adult Sprague-Dawley animals of mixed gender were included in this study. Anaesthesia was induced with Isoflurane (Vedco Inc. St. Joseph, Mo.) by placing the animals into an airtight induction chamber. Surgical anesthesia was induced by intraperitoneal (IP) injection (0.8 mL/100 g) of a combination of urethane (800 mg/kg; Sigma-Aldrich Co., MO) and alpha-chloralose (80 mg/kg; Acros Organics, NJ). Once anaesthetized, body temperature was maintained using a heating pad (HTP-1500 w/ST-017 Soft-Temp Pad, Adroit Medical Systems, TN). Supplemental IP injections of urethane/alpha-chloralose were administered as needed to maintain anaesthesia at a surgical plane. The left femoral artery was exposed and catheterized using a short length (10 mm) of PE-100 tubing filled with heparinized saline (30 U/mL). A midline incision on the ventral side of the animal was used to obtain access to and visualization of the left carotid artery and left cervical vagus. Finally, a tracheostomy tube was inserted through an incision in the trachea to facilitate mechanical ventilation in case the animal stopped breathing.

B. Electrode Configuration and Nerve Stimulation

Two sets of platinized Pt—Ir bipolar hook electrodes (800-micrometer anode/cathode spacing, FHC, Bowdoin, Me.) were positioned on the exposed left cervical vagus nerve as shown in FIG. 5. The left cervical vagus was crushed using a pair of forceps rostral to rostral hook electrode to eliminate rostrally directed reflex responses due to electrical stimulation.

Needle electrodes were applied to the chest of the animal to monitor ECG. The ECG signal was band-pass filtered (Highpass: 0.1 Hz; Lowpass: 300 Hz) and amplified (1000×gain) via a DP-311 differential amplifier (Warner Instruments, Hamden, Conn.). Blood pressure was encoded into a voltage equivalent by a calibrated voltage transducer (Radnoti, Monrovia, Calif.).

C. Experimental Paradigm

Standard rectangular pulse trains consisting of 10 pulses (1 ms PW) at 25 Hz repeated at 1 Hz were applied to the vagus nerve using a opto-isolated stimulator (Digitimer LTD DS3) triggered by a pulse generator (Hewlett Packard 33120A) at an adequate level to evoke bradycardia and hypotension. Without block, the stimulus results in a heart rate drop from ˜5 Hz to ˜1 Hz and a concomitant drop in mean blood pressure from 90-110 mmHg to less than 50 mmHg. When the blood pressure below ˜50 mmHg vagal stimulation was discontinued to enable the blood pressure to return to its normal set point. The LFACb waveform was generated using a dual channel waveform generator (Rigol DG5072) coupled to an isolated voltage controlled current source (Stanford Research Systems Model CS580). Adequate block amplitude was determined using a 1 Hz sinusoidal waveform and increasing the amplitude of the waveform until the effect of the vagal stimulation was blocked. Nominally, the block current was ˜100 pAp (current to peak) corresponding to a voltage drop across the electrode of between 1-2 Vp.

To test the effect of the LFACb, the vagal stimulus train and the LFACb waveform were presented in a regular continuous sequence as follows:

(1) 20 s baseline period of no stimulation (Pre)

(2) 20 s LFACb delivered to the CE (LFAC only)

(3) 20 s LFACb at the CE and vagal stimulation at the RE (LFAC+VStim)

(4) Vagal stimulation at RE (Vstim_Only) until BP falls below ˜50 mmHg

(5) No stimulation return to baseline (Post)

This test sequence was repeated 3× followed by 3× of a control case where the vagal stimulation was applied to the CE and LFACb to the RE.

The ECG and BP along with the LFACb waveform and voltage drop across the LFACb electrode were continuously recorded at 10 kHz via a NI USB DAQ 6212 (National Instruments, Austin, Tex.) using Mr. Kick III (Aalborg, Denmark).

D. Data Analysis

The analysis of the acquired data sets was performed using custom software written for Matlab (Mathworks, Natick, Mass.). The continuously acquired ECG and BP were segmented into 5 epochs corresponding to the conditioning sequence, and identified as follows: PRE, LFAC only, LFAC+VSTIM, VSTIM only, POST. The R—R rate (RRrate) during each condition and the median RRrate for each segment was calculated. The percent block during each experimental segment was calculated using the following equation:

$$\% Block = \frac{[ cond ] - median ( {RRrate}_{pre} )}{median ( {RRrate}_{pre} ) - median ( {RRrate}_{stim} )} * 100$$

Results

The trains of vagal stimulation induced an episodic reduction in heart rate which presented as an increase in the RR interval with dropped heart beats (FIG. 3). These resulted in a smoother drop in blood pressure. Since the major effect were the minima in RR rates during vagal stimulation alone, the RR rates were calculated and the local minima in rate associated with dropped heart beats were used to quantify the effect of the vagal stimulation without block.

FIG. 2 is a representative example of the change in ECG and blood pressure as a function of stimulation condition. It shows that LFAC alone does not alter the ECG rhythm or waveform. When LFAC is used during vagal stimulation, the ECG rhythm shows little to no change. Once LFAC is removed, there is a rapid disruption in the heart rhythm. When vagal stimulation is removed, the heart rhythm returns to its initial state after a slight overshoot likely due to sympathetic rebound. The blood pressure, follows the same trend as the RR rate with little or no change except during the case where vagal stimulation is presented alone.

Taking the local minima of the RR rate and using the prevention of disruption to the heart rhythm as a biomarker, the percent block was estimated (FIG. 4). The absence of RR rate depression during LFAC+VStim suggests that LFAC blocked the effects of vagal stimulation projecting to the heart. In this particular example, LFAC achieved a 98.5±2.5% block of the effects of vagal stimulation.

A possible explanation of the apparent block is if LFAC is activating the nerve and blocking the vagal stimulation volley through collision block. As a control, the vagal stimulation and LFAC sites were reversed such that VStim was presented on the caudal electrode and LFAC was presented on the rostral electrode. If collision block is the mechanism of the block, reversing the electrodes should also result in a block in the LFAC+VStim case. If this is not the mechanism, then the LFAC+VStim case should result in a depression of the heart rate. A typical result of this control case is shown in FIG. 5. Swapping the electrodes in the control case results in 2.9% block, suggesting that the effects of vagal stimulation is not blocked and discounting the possibility that LFAC block is due to collision block.

The results of all 6 rats are presented below in Table 1.

TABLE 1
Vagal stimulation and LFAC waveform parameters
used in the set of 6 rats in this study.

| | Vagal Stimulation | | LFAC waveform | | |
|---|---|---|---|---|---|
| Rat ID | PW (μs) | PA (μA) | Current (μAp) | Freq (Hz) | % Block |
| Rat 46 | 100 | 270 | 160 | 1 | 83.0 |
| Rat 53 | 1000 | 63 | 2.5* | 1 | 60.3 |
| Rat 55 | 1000 | 29 | 100 | 1 | 83.1 |
| Rat 56 | 1000 | 20.75 | 75 | 1 | 100.0 |
| Rat 57 | 1000 | 19.5 | 75 | 1 | 68.1 |
| Rat 58 | 1000 | 290 | 82.5 | 1 | 95.1 |
| Mean | 850.0 | 115.4 | 98.5 | | 81.6 |
| SD | 367.4 | 128.6 | 35.9 | | 15.2 |

On average LFAC resulted in =82% block of the effects of vagal stimulation. The LFAC waveform was well below the currents needed to achieve kHFAC block. In one case, the instrumentation had connection issues which prevented currents >2.5 uA from being presented to the electrode. Despite the limitation, 60% block was achieved.

### IV. Discussion

In this work a low frequency alternating current waveform at 1 Hz and current levels less than 200 μAp was sufficient to achieve >80% block of the effects of descending activity generated by vagal stimulation. The LFAC waveforms were well within the water window and did not cause any apparent injury to the nerve. The effects were immediate without onset activation and immediately reversed when the waveform was discontinued. These initial observations suggest that LFAC block is a potentially biocompatible means to achieve reversible block of conducting nerve activity. Our companion paper suggests that the mechanism of block is due to closed state Na+ channel inactivation. Moreover, the block could be tunable to nerve fiber caliber and type. However, translation to larger nerves will require more work to optimize the waveform and electrode.

An Implantable Neurostimulation System

FIG. 6 shows a diagram of one embodiment of an implantable neurostimulation system 100 according to the invention.

The neurostimulation system 100 comprises a neural interface device 102 for stimulating and/or inhibiting neural activity in a nerve such as the cervical vagus nerve (not shown). Example neural interface embodiments may comprise of neural cuffs that fully or partially circumferentially enclose a segment of the nerve. The system may be used on any nerve that produces a physiological response when suitably stimulated. Examples include the cervical vagus nerve (which may produce an increase or decrease in heart rate) and the splenic nerve (which may produce an increase or decrease in blood pressure).

In other embodiments two or more neural interface devices may be provided, and any plurality of such neural interface devices may be separate or coupled. The neural interface devices may be in the form of a cuff, or any other interface suitable for attaching to or being positioned adjacent a nerve.

The neural interface device 102 comprising first and second electrodes 104, 106. Where two or more neural interface devices are provided, each may have one or more electrodes. For instance, a system may comprise first and second neural interface devices, wherein the first neural interface device comprises a first electrode, and the second neural interface device comprises a second electrode. In some embodiments, the ‘first electrode’ may be a pair of ‘first electrodes’ such that a bipolar signal can be applied across the electrodes in the pair. Likewise, the ‘second electrode’ may be a pair of ‘second electrodes’ such that a bipolar signal can be applied across the electrodes in the pair. Thus, in one embodiment, the neural interface device may comprise four electrodes; i.e. two pairs. In another embodiment, the first and second electrodes may share a common third electrode which is again used to apply bipolar signals between the first electrode and the common third electrode, and between the second electrode and the common third electrode. In another embodiment, first and second electrodes are used and the signals are monopolar.

The neurostimulation system 100 comprises signal generator 108 electrically coupled to the first and second electrodes 104, 106. The signal generator 108 is configured to generate a first, simulation signal which it applies to the nerve to which the neural interface device 102 is attached via the first electrode 104. The first stimulation signal may be the Vstim signal, as described above, or an equivalent signal. In any case, the first stimulation signal is configured to stimulate neural activity in the nerve to cause at least one pre-determined physiological response, such as a drop in heart rate or drop in blood pressure, as described above. However, embodiments of the system may be configured such that any pre-determined physiological response is utilised, depending on the nerve on which the neurostimulation system 100 is used, and the signal that is applied. Examples include a rise or fall in heart rate, a rise or fall in respiratory rate, a rise and fall in blood pressure, and so on. It will be appreciated that for use in humans a de minimis response is desired, and in particular a response which does not affect the well-being of the human.

The signal generator 108 is configured to generate a second, blocking signal which it applies to the nerve to which the neural interface device 102 is attached via the second electrode 106. The second blocking signal may be the LFAC signal, as described above, or an equivalent signal. In any case, the second blocking signal is configured to stimulate neural activity in the nerve to cause a partial or complete block in the neural activity in the nerve. In particular, the second blocking signal is configured to block (i.e. at least partially, optionally fully, suppress) the pre-determined physiological response caused by the first signal. It will be appreciated that in order to block effectively, the pulses of the blocking signal must overlap with the pulses of the stimulating signal. By ‘overlap’, it is mean that the effects of the pulses are temporarily correlated so as to counteract each other in the neural activity of the nerve.

The neurostimulation system 100 further comprises a physiological sensor 110 to detect the at least one pre-determined physiological response. In some cases, a plurality of such sensors may be used, which may sense the same or different physiological responses. Exemplary sensors include a heart rate sensor, a blood pressure sensor and a sensor for detecting respiratory rate.

The neurostimulation system 100 further comprises a control system 112 communicatively coupled to the physiological sensor 110. The function of the control system 112 is to generate a feedback response upon receiving a signal from the physiological sensor. The nature of the feedback response can differ. For example, the feedback response may indicate that the second, blocking signal delivered via the second electrode 106 is effective. The control system 112 would be capable of determining this by reference to the signal from the physiological sensor. For example, if the first stimulating signal is being applied by the signal generator 108 but the predetermined physical response that would be expected is not happening because of the application of the second, blocking signal by the signal generator, then the control system can determine that the blocking signal is effective. As a consequence, the control system may issue a notification (for instance, to a user interface device across a wireless connection) that the implant is operating effectively. Conversely, if the first stimulating signal is being applied by the signal generator 108 and the predetermined physical response that would be expected is continuing to happen despite the application of the second, blocking signal by the signal generator, then the control system can determine that the blocking signal is not effective. For completeness, it should be noted that the physiological response may be happening, but at a reduced or increased rate compared with the ideal, in which case it may be inferred that the blocking signal is partially effective.

With reference to FIGS. 2 and 3, it is possible to determine between seconds 40 and 50, for example that the second, blocking signal LFAC is effective in blocking the first stimulating signal VStim because the predetermined physiological responses of a drop in heart rate and blood pressure (which can be seen immediately after 50 seconds, when the blocking signal LFAC is not applied) are not happening.

Where the control system determines that the blocking signal is not effective or partially effective, the control system may cause the signal generator to alter the signal parameters of the second, blocking signal. Suitable signal parameters for adjustment include amplitude, phase, frequency, waveform shape and so on.

Again, with reference to the examples mentioned above, it may be inferred by reference to the physiological sensor (i.e. the heart rate or blood pressure sensors) that application of the second blocking signal, LFAC, is effective in partially blocking the first stimulation signal, VStim, (i.e. it is partially effective) because the sensed heart rate is reduced to a lesser extent compared with the heart rate sensed when the signal generator is not generating the second blocking signal, LFAC, but applying the first stimulation signal, VStim. In this case, the controller may increase the amplitude of the second blocking signal, LFAC, to achieve a more complete or full block.

The control system 112 may be configured to apply the first and second signals independently and concomitantly in any order to determine whether the second, blocking signal is effective. For example, in the examples mentioned above a sequence is described where a controller would apply:
(1) no stimulation via either the first or second electrodes [i.e. not generating the first or second signals] for a period of 20 seconds
(2) stimulation via the second electrode only [i.e. generating the second, blocking signal without generating the first, stimulating signal] for a period of 20 seconds
(3) stimulation via the first and second electrodes concomitantly [i.e. generating the first, stimulating signal and the second, blocking signal concomitantly] for a period of 20 seconds
(4) stimulation via the first electrode only [i.e. generating the first, stimulating signal without generating the second, blocking signal] until the physiological response reaches a threshold.
(5) no stimulation via either the first or second electrodes [i.e. not generating the first or second signals] until the physiological baseline has been restored.

It will be appreciated that the 20 seconds duration is merely exemplary, and any suitable time period may be used, such as between 1 and 60 second, preferably 5 and 40 seconds, preferably between 10 and 30 second.

It will be appreciated that the sequence described above is purely exemplary, and other sequences are possible depending on circumstances. For example, the controller may be configured to apply the following sequence:
(1) no stimulation via either the first or second electrodes [i.e. not generating the first or second signals] for a period of 20 seconds
(2) stimulation via the first electrode only [i.e. generating the first, stimulating signal without generating the second, blocking signal] until the physiological response reaches a threshold.
(3) stimulation via the first and second electrodes concomitantly [i.e. generating the first, stimulating signal and the second, blocking signal concomitantly] for a period of 20 seconds, or until the physiological baseline has been restored.
(4) if physiological baseline is not restored, no stimulation via either the first or second electrodes [i.e. not generating the first or second signals] until the physiological baseline has been restored.

Other sequences are also possible.

In determining the feedback response, the control system 112 is configured to compare the physiological signal sensed by the physiological sensor in the periods mentioned above, for example by comparing signal comparators that are representative of the signal sensed by the physiological sensor at the time.

It will be appreciated that the invention has been described with reference to specific examples and the scope of the invention is not limited to those specific examples but is as defined in the appended claims.

### REFERENCES

[1] D. M. Ackermann, N. Bhadra, E. L. Foldes, and K. L. Kilgore, “Conduction block of whole nerve without onset firing using combined high frequency and direct current,” Medical & biological engineering & computing, vol. 49, pp. 241-251, Oct. 2, 2011.
[2] N. Bhadra and K. L. Kilgore, “Direct current electrical conduction block of peripheral nerve,” IEEE Trans Neural Syst Rehabil Eng, vol. 12, pp. 313-24, September 2004.
[3] N. Bhadra and K. L. Kilgore, “High-frequency electrical conduction block of mammalian peripheral motor nerve,” Muscle & Nerve: Official Journal of the American Association of Electrodiagnostic Medicine, vol. 32, pp. 782-790, 2005.
[4] K. L. Kilgore and N. Bhadra, “Reversible nerve conduction block using kilohertz frequency alternating current,” Neuromodulation, vol. 17, pp. 242-54; discussion 254-5, April 2014.
[5] M. Sassen and M. Zimmermann, “Differential blocking of myelinated nerve fibres by transient depolarization,” Pflugers Arch, vol. 341, pp. 179-95, Jul. 6, 1973.
[6] M. Franke, T. Vrabec, J. Wainright, N. Bhadra, N. Bhadra, and K. Kilgore, “Combined KHFAC+DC nerve block without onset or reduced nerve conductivity after block,” J Neural Eng, vol. 11, p. 056012, October 2014.
[7] J. Tiede, L. Brown, G. Gekht, R. Vallejo, T. Yearwood, and D. Morgan, “Novel spinal cord stimulation parameters in patients with predominant back pain,” Neuromodulation, vol. 16, pp. 370-5, July-August 2013.
[8] D. M. Ackermann, N. Bhadra, E. L. Foldes, X.-F. Wang, and K. L. Kilgore, “Effect of nerve cuff electrode geometry on onset response firing in high-frequency nerve conduction block,” IEEE Transactions on Neural Systems and Rehabilitation Engineering, vol. 18, pp. 658-665, 2010.
[9] T. Vrabec, N. Bhadra, J. Wainright, N. Bhadra, M. Franke, and K. Kilgore, “Characterization of high capacitance electrodes for the application of direct current electrical nerve block,” Medical & biological engineering & computing, vol. 54, pp. 191-203, 2016.
[10] S. B. Brummer and M. J. Turner, “Electrical stimulation with Pt electrodes: II-estimation of maximum surface redox (theoretical non-gassing) limits,” IEEE Trans Biomed Eng, vol. 24, pp. 440-3, September 1977.
[11] N. J. Rijkhoff, H. Wijkstra, P. E. van Kerrebroeck, and F. M. Debruyne, “Selective detrusor activation by electrical sacral nerve root stimulation in spinal cord injury,” J Urol, vol. 157, pp. 1504-8, April 1997.
[12] J. G. Whitwam and C. Kidd, “The use of direct current to cause selective block of large fibres in peripheral nerves,” Br J Anaesth, vol. 47, pp. 1123-33, November 1975.
[13] S. B. Brummer and M. J. Turner, “Electrochemical considerations for safe electrical stimulation of the nervous system with platinum electrodes,” IEEE Trans Biomed Eng, vol. 24, pp. 59-63, January 1977.
[14] E. L. Foldes, D. Ackermann, N. Bhadra, and K. L. Kilgore, “Counted cycles method to quantify the onset response in high-frequency peripheral nerve block,” Conf Proc IEEE Eng Med Biol Soc, vol. 2009, pp. 614-7, 2009.
[15] J. D. Miles, K. L. Kilgore, N. Bhadra, and E. A. Lahowetz, “Effects of ramped amplitude waveforms on the onset response of high-frequency mammalian nerve block,” J Neural Eng, vol. 4, pp. 390-8, December 2007.
[16] G. C. Santa Cruz Chavez, B.-Y. Li, P. A. Glazebrook, D. L. Kunze, and J. H. Schild, “An afferent explanation for sexual dimorphism in the aortic baroreflex of rat,” American Journal of Physiology—Heart and Circulatory Physiology, vol. 307, pp. H910-H921, 2014.

1.-28. (canceled)

29. An implantable neurostimulation system, comprising:
at least one neural interface device for stimulating and/or inhibiting neural activity in a nerve such as the cervical vagus nerve, the at least one neural interface device comprising:
first and second electrodes;
at least one signal generator electrically coupled to the first and second electrodes and configured to generate first and second electrical signals that stimulate and/or inhibit neural activity in the nerve with the first and second electrical signals via the first and second electrodes, respectively;
wherein the first electrical signal is configured to stimulate neural activity in the nerve to cause at least one pre-determined physiological response; and
wherein the second electrical signal is configured to inhibit neural activity in the nerve to at least partially, optionally fully, suppress the least one pre-determined physiological response;
the implantable neurostimulation system further comprising:
at least one physiological sensor to detect the at least one pre-determined physiological response; and
a control system communicatively coupled to the at least one physiological sensor and configured to generate a feedback response, upon receiving a signal from at least one physiological sensor.

30. The system of claim 29, wherein the control system is configured to cause the signal generator to generate the first signal to stimulate neural activity in the nerve at a first time, and to cause the signal generator to generate the second signal to inhibit neural activity in the nerve at a second time later than the first time and concomitantly with the first signal.

31. The system of claim 29, wherein the control system is configured to cause the signal generator to generate the second signal to inhibit neural activity in the nerve at a first time, and to cause the signal generator to generate the first signal to stimulate neural activity in the nerve at a second time later than the first time and concomitantly with the second signal.

32. The system of claim 30, wherein the control system is configured to generate a feedback response upon receiving a signal from the at least one physiological sensor whilst the second signal is being generated concomitantly with the first signal.

33. The system of claim 30, wherein the control system is configured to compare a first signal comparator representative of a signal from the at least one physiological sensor whilst the second signal is being generated concomitantly with the first signal, with a second signal comparator representative of a signal from the at least one physiological sensor whilst the first signal is being generated without the second signal.

34. The system of claim 33, wherein the control system is configured to generate a feedback response based on a comparison of the first signal comparator with the second signal comparator.

35. The system of claim 29, wherein the feedback response is indicative of the effectiveness of the second electrical signal to inhibit neural activity and thus to at least partially, optionally fully, suppress the least one pre-determined physiological response.

36. The system of claim 35, wherein the feedback response is indicative of the pre-determined physiological response being fully suppressed.

37. The system of claim 35, wherein the feedback response is indicative of the pre-determined physiological response being partially suppressed.

38. The system of claim 35, wherein the feedback response is indicative of the pre-determined physiological response not being suppressed.

39. The system of claim 38, wherein the control system is configured to modify the second signal based on a feedback response indicative of the pre-determined physiological response being partially suppressed or not being suppressed.

40. The system of claim 29, wherein the at least one pre-determined physiological response is a change in heart rate, respiratory rate, and/or blood pressure.

41. The system of claim 40, wherein the at least one physiological sensor is a heart rate sensor, a sensor for detecting respiratory rate, and/or a blood pressure sensor, respectively.

42. The system of claim 29, wherein the first electrode is located either closer to or further away from the brain along the nerve axis than the second electrode.

43. The system of claim 29, wherein the first electrical signal comprises a pre-determined pattern that causes the pre-determined physiological response.

44. The system of claim 30, wherein the control system is configured to generate the second signal concomitantly with the first signal for a first period.

45. The system of claim 44 wherein the control system is configured to generate the second signal without the first signal for a second period prior to the first period.

46. The system of claim 44 wherein the control system is configured to generate the first signal without the second signal for a third period following the first period.

47. The system of claim 44 wherein the control system is configured to generate the second signal without the first signal for a second period following the first period.

48. The system of claim 44 wherein the control system is configured to generate the first signal without the second signal for a third period prior to the first period.
